(19) United States
(12) Patent Application Publication
(10) Pub. No.: US 2026/0364275 A1
(43) Pub. Date: Nov. 19, 2026

(54) ULTRASONIC SENSOR FOR DETECTING AIR BUBBLES IN INFUSION FLUIDS

(71) Applicant: B. Braun Melsungen AG, Melsungen (DE)

(72) Inventors: Ernst Bressau, Malsfeld (DE); Rolf Hollstein, Alheim-Heinebach (DE)

(21) Appl. No.: 19/678,770

(22) Filed: May 15, 2026

(30) Foreign Application Priority Data
May 19, 2025 (EP) 25177362.8

(51) Int. Cl. A61M 5/36; A61M 5/142; G01N 29/02; G01N 29/26

(52) U.S. Cl. CPC A61M 5/365; A61M 5/14232; G01N 29/02; G01N 29/26; A61M 2205/3375; G01N 2291/021; G01N 2291/102

(57) ABSTRACT

An air bubble sensor is configured for use with an infusion pump. The air bubble sensor includes an ultrasonic transmitter, an ultrasonic receiver, and a tubing holder that extends along a substantially horizontal conveying direction. The ultrasonic transmitter is located on a first side of the tubing holder, or it forms a first side of the tubing holder. The ultrasonic receiver is located on a second side opposite the first side, or it forms a second side of the tubing holder. A measurement path extending from the ultrasonic transmitter to the ultrasonic receiver is not vertical or perpendicular, but is oriented at an angle of up to 45°, preferably up to 25°, relative to the horizontal plane.

### CROSS-REFERENCE TO RELATED APPLICATION

This application claims priority under 35 U.S.C. § 119 to European Application No. 25177362.8, filed on May 19, 2025, the content of which is incorporated by reference herein in its entirety.

### FIELD

The present disclosure relates to air bubble detection and, more specifically, to the preferably additional detection of particles in a medical infusion fluid within an infusion line, particularly within the infusion tubing of an infusion pump, using an ultrasonic sensor. More specifically, the present disclosure relates to an ultrasonic sensor and an infusion pump equipped with an ultrasonic sensor.

### BACKGROUND

In medicine, liquid medications are administered to patients using infusion pumps and injected into their bloodstream either continuously or intermittently. It is important that no air bubbles (or other gas bubbles) are introduced together with the liquid medication. The liquid medication is referred to below as the infusion fluid.

The applicant’s European Patent 3 296 717 A1 discloses an optical imaging sensor located on the outer circumference of a tubing in a dialysis machine, wherein the sensor is designed to detect foreign substances such as contaminants and air bubbles in the tubing.

Peristaltic infusion pumps are known in the prior art in which a tubing section of an infusion tubing is inserted in a straight line into the tubing holder(s). The so-called peristaltic mechanism consists of several external, radially movable displacement elements that are sequentially pressed into the elastic tubing (or a specially formed elastic pump segment) of the infusion tubing in such a way that the infusion fluid is propelled forward. An ultrasonic sensor for detecting air bubbles is located next to the peristaltic mechanism.

In the prior art, such infusion pumps have a front flap on the front loading side, which is opened before the relevant section of the infusion tubing is inserted horizontally into several forward-opening tubing holders (tubing mounts). The front access panel of the infusion pump is then closed with the front flap, which helps to hold the relevant section of the infusion tubing securely in place within the housing.

The air bubble sensor, located directly behind the front flap, has an ultrasonic transmitter and an ultrasonic receiver on the outer circumference of the tubing. In accordance with the horizontal insertion direction of the infusion tubing described above, the transmitter and receiver are arranged vertically, one above the other, in the direction of gravity. According to the prior art, this results in a perpendicular ultrasonic measurement path running through the center of the infusion tubing.

In particular, in the horizontal section of the infusion tubing, so-called trapped air bubbles can become lodged against the inner wall of the tubing and remain there for a significant amount of time without being carried along by the flowing infusion fluid. This process may continue, for example, until another air bubble (cleaning bubble), moving along with the flowing infusion fluid, carries the stationary air bubble away.

Since the air or other gas that forms the bubble has a lower density than the infusion fluid, air bubbles typically form at the top of the infusion tubing, that is, near the top of the inner wall of the tubing.

If a stationary air bubble (by chance) becomes lodged between the ultrasonic transmitter and the ultrasonic receiver, the infusion pump’s electronics or control unit incorrectly interprets the resulting sustained attenuation of the ultrasonic signal as an indication that a large amount of air is present in the infusion fluid flowing to the patient, thereby triggering a false alarm.

### SUMMARY

The objective of this disclosure is to provide an ultrasound-based air bubble sensor and an infusion pump equipped with such an air bubble sensor, in which air bubble detection is improved. In particular, false alarms caused by a non-critical stationary air bubble in the measurement channel between the ultrasonic transmitter and the ultrasonic receiver should be avoided.

The air bubble sensor described in the disclosure includes an ultrasonic transmitter, an ultrasonic receiver, and a tubing holder designed and configured to accommodate an infusion tubing. The tubing holder extends primarily along a roughly horizontal conveying direction; thus, the tubing holder is angled, for example, by a maximum of 5° from the horizontal. In other words, the tubing holder is designed so that a tubing inserted into it is aligned horizontally. The adjective “horizontal” refers to the Earth’s gravitational field. This arrangement of the tubing holder and the infusion tubing that fits into it raises the issue of air bubbles that may be present in the infusion tubing. The ultrasonic transmitter is located on one side of the tubing holder and/or forms one side of the tubing holder. The ultrasonic receiver is located on a second side of the tubing holder opposite the first side and/or forms this second side of the tubing holder. In this design, the ultrasonic transmitter and receiver are positioned on the outer circumference of the infusion tubing to be accommodated. A measurement path extending from the ultrasonic transmitter to the ultrasonic receiver is not vertical or perpendicular (to the Earth's surface), but is preferably inclined at a maximum angle of 45°, and more preferably at a maximum angle of 25°, relative to the horizontal plane. This means that the measurement path is not aligned with the full diameter or the entire length of an air bubble (which is not too large) located at the top of the infusion tubing, but rather, at most, with a lower secant of that air bubble. It is also possible that the measurement path merely touches the air bubble or even passes on the outside of it. As a result, the portion of the measurement path that passes through the (not overly large) stationary air bubble is shortened – preferably minimized – (compared to a measurement method using the prior art). This reduces the damping detected by the air bubble sensor. As a result, the reading may fall below the alarm threshold of an electronic device or control unit that evaluates the signal from the air bubble sensor. This helps prevent false alarms caused by air bubbles in the air bubble sensor’s measurement path that are not critical to the patient.

Of course, the air bubble sensor described in the disclosure is also designed to detect air in the infusion tubing even when there is no infusion fluid in the measurement path – for example, when no infusion fluid is being delivered at all due to an operating error. In that case, the damping value determined by the air bubble sensor is, of course, above the alarm threshold.

The term “air bubbles” also refers to bubbles of other gases.

Preferably, the air bubble sensor is also designed to detect particles that must not be infused into the patient’s bloodstream.

False alarms caused by air bubbles in the air bubble sensor’s measurement path can be effectively prevented if the measurement path is approximately horizontal (with a maximum deviation of 5°).

In preferred embodiments of the air bubble sensor, the underside of the tubing holder is formed by a tubing support on which the infusion tubing can be placed. In particular, the tubing support is positioned below the measurement path at a distance equal to the radius of the outer jacket of the infusion tubing.

In one embodiment, the ultrasonic transmitter is movable relative to the ultrasonic receiver, which is fixed to the housing, or the ultrasonic receiver is movable relative to the ultrasonic transmitter, which is fixed to the housing.

In that case, one side of the tubing holder may be formed by a tubing assembly, so that the tubing holder is closed on three sides and open on one side. When installing the tubing in a peristaltic infusion pump, this means that the tubing holder is open at the front.

In another embodiment, the ultrasonic transmitter and the ultrasonic receiver are both mounted to the housing and fixed relative to one another.

To this end, the ultrasonic transmitter and the ultrasonic receiver may be connected to each other by means of the tubing support positioned beneath the infusion tubing, so that the tubing holder is closed on three sides and open at the top.

In an exemplary embodiment featuring an inclined measurement path – for example, a measurement path inclined at an angle of 25 to 45 degrees relative to the horizontal – the ultrasonic transmitter and the ultrasonic receiver are connected to each other via a connecting element that is also inclined. This also ensures that the ultrasonic transmitter and receiver are securely attached to the housing of the infusion pump in question, and that the tubing holder is enclosed on three sides. In particular, the connecting element is positioned at a distance from the measurement path corresponding to the radius of the outer wall of the infusion tubing.

The medical infusion pump described in the disclosure has a housing and an air bubble sensor as described in the disclosure (or as described above). The housing or the feet attached to it define a support plane to which the conveying direction is roughly parallel, i.e., at an angle of no more than 5°. Also in relation to the support plane, the measurement path is not perpendicular or vertical, but is preferably inclined at a maximum angle of 45°, and more preferably at a maximum angle of 25°, relative to the support plane. Consequently, when the infusion pump is mounted in the usual horizontal position (e.g., in a rack), the measurement path does not pass through the full diameter of an air bubble located at the top of the infusion tubing, but rather, at most, through a lower secant of the air bubble. It is also possible that the measurement path touches or passes through the stationary air bubble. As a result, the portion of the measurement path that is attenuated by the stationary air bubble is shortened – and preferably minimized – compared to a measurement using the prior art. As a result, the value may fall below the alarm threshold of the infusion pump’s electronic system or control unit, which evaluates the signal from the air bubble sensor. This prevents false alarms caused by air bubbles in the air bubble sensor's measurement path.

False alarms caused by trapped air bubbles can be effectively prevented if the measurement path is roughly parallel to the support plane, i.e., if it is angled at a maximum of 5° relative to the support plane of the infusion pump.

The infusion pump may be a peristaltic pump, meaning it uses a peristaltic mechanism. A section of the infusion tubing is inserted straight into such an infusion pump, against which several outer, radially movable displacement elements of the peristaltic mechanism rest. The infusion fluid is conveyed by the displacement elements, which move radially in succession.

The peristaltic mechanism is preferably covered by a front flap, wherein the section of the infusion tubing within the infusion pump can be securely fixed to the housing or held in place by the closed front flap.

In an exemplary embodiment of the peristaltic infusion pump, the ultrasonic transmitter is formed on or attached to the front flap, while the ultrasonic receiver is formed as an integral part of the housing (e.g., on the front of the pump housing) or attached thereto.. Alternatively, in a kinematic reversal, the ultrasonic receiver is formed on or attached to the front flap, while the ultrasonic transmitter is formed as an integral part of the housing (e.g., on the front of the pump) or attached to it.

In another exemplary embodiment of the peristaltic infusion pump, both the ultrasonic transmitter and the ultrasonic receiver are fixed to the housing. When the front flap is closed, the ultrasonic receiver or the ultrasonic transmitter is positioned adjacent to the front flap. In that case, the tubing holder is open upward or at an upward angle.

The infusion pump may be a roller pump. A section of the infusion tubing is inserted into such an infusion pump along a curved path. In the curved section, one or more outer rollers move along the length of the tubing section, thereby conveying the infusion fluid. For this type of pump, the air bubble sensor described above, with the tubing holder facing upward, is the preferred choice.

For both types of pumps, the air bubble sensor is preferably located downstream of the peristaltic mechanism or the rollers, or on the patient side.

### BRIEF DESCRIPTION OF THE DRAWINGS

FIG. 1 shows a first exemplary embodiment of an infusion pump according to the present disclosure;

FIG. 2 shows a schematic representation of a portion of the infusion pump from FIG. 1, incorporating a state-of-the-art air bubble sensor;

FIG. 3 shows a portion of the infusion pump from FIG. 1, including a first exemplary embodiment of an air bubble sensor according to the present disclosure, in a schematic representation;

FIG. 4 shows a portion of the infusion pump from FIG. 1, including a first variant of a second exemplary embodiment of an air bubble sensor according to the present disclosure, in a schematic representation;

FIG. 5 shows a substantial portion of a second exemplary embodiment of an infusion pump according to the present disclosure in a schematic representation;

FIG. 6 shows the signal curves of the state-of-the-art air bubble sensor shown in FIG. 2, the air bubble sensors shown in FIGS. 3 and 4, and an air bubble sensor according to a further exemplary embodiment; and

FIG. 7 shows a second variant of the second exemplary embodiment of the air bubble sensor according to the present disclosure in a schematic representation.

### DETAILED DESCRIPTION

The prior art and exemplary embodiments of the present disclosure are described below with reference to the accompanying figures.

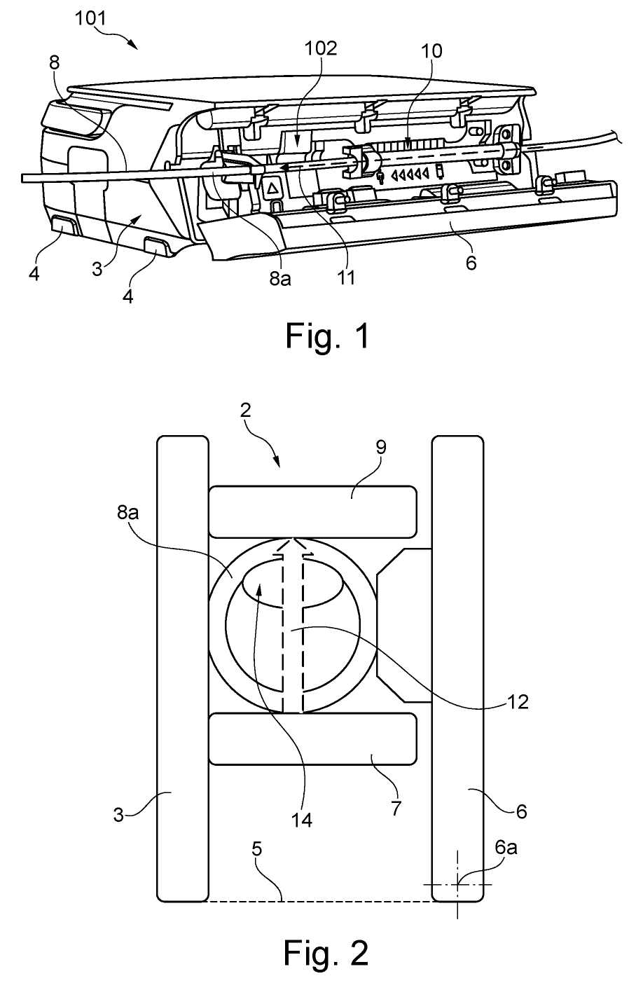

FIG. 1 shows a first exemplary embodiment of an infusion pump 101 according to the present disclosure. As is known from the prior art, it has a housing 3 with four feet 4 that define the support plane of the infusion pump 101. In the normal operating position of the infusion pump 101, this support plane is horizontal, i.e., perpendicular to the Earth's gravitational field.

A front flap 6 (shown open in FIG. 1) is mounted on the front side of housing 3, as shown in FIG. 1. Behind this front flap 6, a section 8a of an infusion tubing 8 extends across the full width of the housing 3 or the front flap 6, wherein this tubing section 8a comprises a so-called pump segment made of softer silicone. In this section of the pump, the displacement elements of a peristaltic mechanism 10 engage to convey the infusion fluid in the infusion tubing 8 (to the left in FIG. 1) along a conveying direction 11 toward a first air bubble sensor 102 and then on to a patient access point.

The tubing section 8a held in the infusion pump 101 is parallel to the support plane formed by the feet 4. Therefore, the tubing section 8a and the conveying direction 11 are also horizontal in the normal operating position of the infusion pump 101. This creates the problem that air bubbles can become trapped in tubing section 8a.

FIG. 2 shows a state-of-the-art air bubble sensor 2 that can be installed on the infusion pump 101 shown in FIG. 1. The tubing section 8a shown in cross-section is clamped and secured between the front of the pump housing 4 and the front flap 6 of the housing 4, which can pivot about a pivot axis 6a. Air bubble sensor 2 has an ultrasonic transmitter 7 and an ultrasonic receiver 9. A measurement path 12 from the lower ultrasonic transmitter 7 to the upper ultrasonic receiver 9 is perpendicular to the support plane 5 and thus perpendicular or vertical to the Earth's surface. Furthermore, the measurement path 12 is aligned centrally through the tubing section 8a.

A stationary air bubble 14 has become lodged in the tubing section 8a inserted into the air bubble sensor 2, so that the measurement path 12 passes through the air bubble 14 over the greatest possible portion of its length. As a result, the signal experiences maximum attenuation. This can trigger an alarm on infusion pump 101, which is an unwanted alarm because this condition (a stationary air bubble in the sensor area) is not critical for the patient.

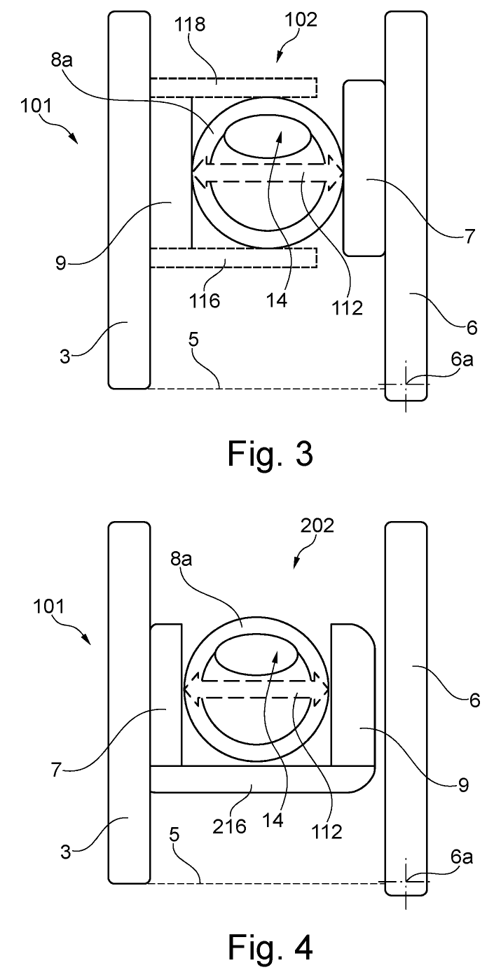

FIG. 3 shows a portion of the housing 3 of the infusion pump 101 from FIG. 1, along with the first exemplary embodiment of the air bubble sensor 102 according to the present disclosure.

Behind the front flap 6 of the housing 4 (shown closed in FIG. 3), which can pivot about pivot axis 6a, the tubing section 8a shown in cross-section is clamped into the tubing holder of the air bubble sensor 102 and secured there. To this end, a tubing holder for the air bubble sensor 102 may include a lower tubing support 116 and an upper tubing assembly 118, with the tubing holder for the air bubble sensor 102 opening toward the front.

The air bubble sensor 102 includes an ultrasonic transmitter 7 and an ultrasonic receiver 9, which, when the front flap 6 is closed, are positioned at the same height and at the same distance from the support plane 5 of the infusion pump 101. The ultrasonic transmitter 7 is mounted on the inside of the front flap 6 and can therefore move around the pivot axis 6a. The ultrasonic receiver 9 is mounted on the front of the pump housing 3. This means that the measurement path 112 is horizontal relative to the support plane 5 and, when the infusion pump 101 is in its normal operating position, is horizontal relative to the ground surface.

Furthermore, in the illustrated exemplary embodiment, the measurement path 112 is aligned centrally through the tubing section 8a, or, in another (unshown) exemplary embodiment, is offset vertically from the center.

FIG. 4 shows a portion of the housing 3 of the infusion pump 101 from FIG. 1, featuring a first variant of a second exemplary embodiment of the air bubble sensor 202 according to the present disclosure. Behind the front flap 6 of the housing 4 (shown closed in FIG. 4), which can pivot about pivot axis 6a, the tubing section 8a shown in cross-section is clamped and secured in the tubing holder.

The tubing holder is open at the top, so that tubing section 8a can be inserted into the tubing holder from above after the front flap 6 is opened and is preferably secured there. By closing the clamp, the tubing section 8a can also be secured to the housing at another location, either as an alternative or in addition.

The air bubble sensor 202 includes an ultrasonic transmitter 7 and an ultrasonic receiver 9, both of which are positioned at the same height and at the same distance from the support plane 5 of the infusion pump 101, such that the measurement path 112 is horizontal/ parallel to the support plane 5 and thus oriented horizontally or parallel to the earth's surface.

The ultrasonic transmitter 7 and the ultrasonic receiver 9 are both mounted on the housing. To this end, they are secured to one another via a connecting element 216, which is located beneath the tubing section 8a and can therefore serve as a tubing support 216.

Furthermore, in the exemplary embodiment shown in FIG. 4, the measurement path 112 is aligned centrally through the tubing section 8a.

As shown in FIGS. 3 and 4, a stationary air bubble 14 has formed in the tubing section 8a used in the air bubble sensor 102; 202, such that the measurement path 112 runs (only) through a lower portion of the air bubble 14, similar to a secant. Consequently, the signal experiences reduced attenuation compared to the prior art, which, according to the disclosure, remains below an alarm threshold of the infusion pump 101, since this condition is not critical for the patient.

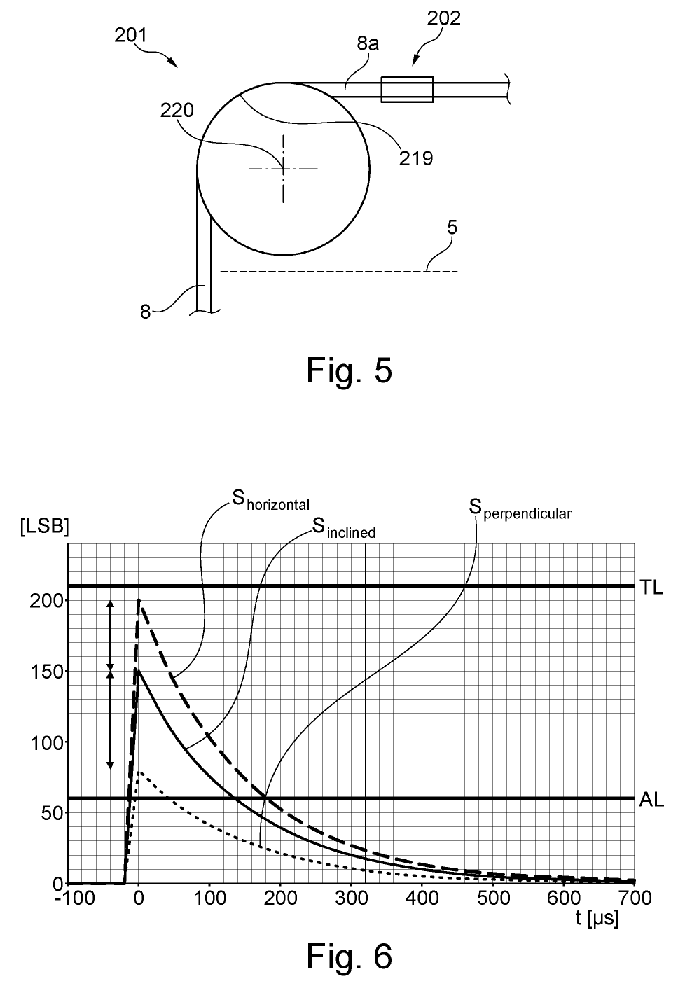

FIG. 5 shows key components of a second exemplary embodiment of an infusion pump 201 according to the present disclosure, incorporating the air bubble sensor 202 shown in FIG. 4. The infusion pump 201 is designed as a roller pump, into which a section 8a of an infusion tubing 8 is also inserted. A portion of the tubing section 8a inserted into the roller pump 201 is bent along a curved trajectory 219. Rollers (not shown) run along the inner surface of the curved portion of the tubing section 8a, rotating about an axis 220. This causes the infusion fluid to flow through that tubing section 8a.

A section of the tubing segment 8a inserted into the roller pump 201, located downstream of the curved portion, extends parallel to the support plane 5 of the housing (not shown in detail) and is therefore positioned horizontally in the normal operating position of the roller pump 201. This section houses the exemplary embodiment of the air bubble sensor 202 shown in FIG. 4, which comprises the housing-mounted ultrasonic transmitter 7 and the housing-mounted ultrasonic receiver 9, and whose tubing holder is open at the top.

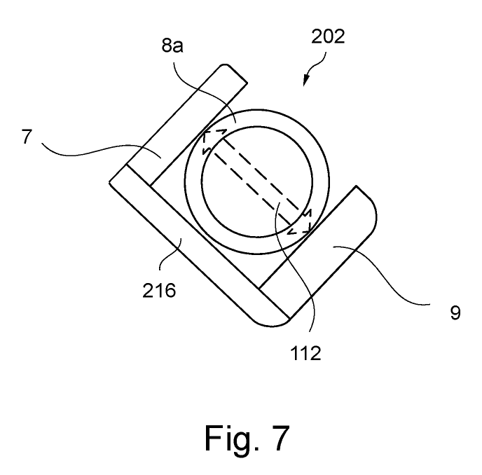

FIG. 6 shows three signal waveforms over time that result when the stationary air bubble 14 shown in FIGS. 2 through 4 is detected by the various air bubble sensors 2, 102, and 202, as shown in FIGS. 1 through 5 and in FIG. 7. More specifically, a comparator input signal S_{perpendicular} of the air bubble sensor 2 of the prior art is shown in FIG. 2. Also shown is a comparator input signal S_{horizontal}of the air bubble sensor 102 is shown in FIG. 3 and the air bubble sensor 202 in FIG. 4. Finally, FIG. 7 shows a comparator input signal S_{inclined}from an air bubble sensor in which the measurement path 12 is tilted by 45°. When the air bubble sensor 102; 202 is rotated 45 degrees, approximately 90% of the air bubble shown is “detected.” In addition, the air bubble sensor 102 shown in FIG. 3 may be tilted or rotated 45° clockwise about a longitudinal axis of the tubing section 8a by 45° relative to the arrangement shown in FIG. 3. Alternatively, the air bubble sensor 202 shown in FIG. 4 is tilted or rotated 45° clockwise about the longitudinal axis of the tubing section 8a, as shown in FIG. 7, relative to the arrangement shown in FIG. 4.

The comparator input signal S_{inclined} also illustrates the reduced attenuation of air bubble sensor 202, as described in the disclosure, according to the second variant of the second exemplary embodiment shown in FIG. 7, compared to air bubble sensor 2 of the prior art.

In particular, the comparator input signal S_{horizontal} illustrates the even further (more significant) reduced attenuation of the air bubble sensors 102, 202, as described in the disclosure, compared to the air bubble sensor 2 of the prior art, in accordance with the exemplary embodiments shown in FIGS. 3 and 4.

FIG. 7 shows a second variant of the second exemplary embodiment of the air bubble sensor 202 according to the present disclosure. Behind the front flap 6 of the housing 4 (shown only in FIG. 4), which can pivot about pivot axis 6a, the tubing section 8a shown in cross-section is received in the tubing holder. The tubing holder is open diagonally upward and forward. The air bubble (shown only in FIG. 4) is at the top (due to its low density).

### LIST OF REFERENCE DESIGNATIONS

2 State-of-the-art air bubble sensor
3 Housing
4 Foot
5 support plane
6 Front flap
6a Pivot axis
7 Ultrasonic transmitter
8 Infusion tubing
8a Tubing section with horizontal conveying direction
9 Ultrasonic receiver
10 Peristaltic mechanism
11 Conveying direction
12; 112 Measurement path
101 Peristaltic infusion pump
102 Air bubble sensor (open to the front)
14 Standing air bubble
116 Tubing support
118 Tubing assembly
201 Infusion pump / roller pump
202 Air bubble sensor (open at the top)
216 Tubing support / connecting element
220 Axis
S_{perpendicular} Signal for a perpendicular measurement path according to the prior art
S_{horizontal} Signal for a horizontal measurement path
S_{inclined} Signal for an inclined measurement path

1. An air bubble sensor comprising:
an ultrasonic transmitter;
an ultrasonic receiver; and
a tubing holder,
the ultrasonic transmitter arranged on a first side of the tubing holder,
the ultrasonic receiver arranged on a second side of the tubing holder opposite the first side of the tubing holder, and
a measurement path directed from the ultrasonic transmitter to the ultrasonic receiver is inclined at an acute angle relative to a horizontal plane.

2. The air bubble sensor according to claim 1, wherein the acute angle is less than or equal to 45°.

3. The air bubble sensor according to claim 1, wherein the acute angle is less than or equal to 25°.

4. The air bubble sensor according to claim 1, wherein a bottom surface of the tubing holder is formed by a tubing support.

5. The air bubble sensor according to claim 4, wherein the ultrasonic transmitter and the ultrasonic receiver are connected to one another via the tubing support, such that the tubing holder is closed on three sides and open at a top side of the tubing holder.

6. The air bubble sensor according to claim 1, wherein the ultrasonic receiver is fixed, and the ultrasonic transmitter is movable relative to the ultrasonic receiver.

7. The air bubble sensor according to claim 1, wherein the ultrasonic transmitter is fixed, and the ultrasonic receiver is movable relative to the ultrasonic transmitter.

8. The air bubble sensor according to claim 1, wherein an upper side of the tubing holder is formed by a tubing assembly, such that the tubing holder is closed on three sides and open on the upper side.

9. The air bubble sensor according to claim 1, wherein the ultrasonic transmitter and the ultrasonic receiver are fixed relative to one another.

10. The air bubble sensor according to claim 1, wherein:
the acute angle is between 25° and 45°, and
the ultrasonic transmitter and the ultrasonic receiver are connected to one another via a connecting element, so that the tubing holder is enclosed on three sides.

11. A medical infusion pump comprising:
the air bubble sensor according to claim 1; and
a housing,
wherein the tubing holder extends along a conveying direction,
housing or feet attached to the housing define a support plane to which the conveying direction is parallel, and
the measurement path is inclined relative to the support plane by the acute angle.

12. The medical infusion pump according to claim 11, wherein the acute angle is less than or equal to 45°.

13. The medical infusion pump according to claim 11, wherein the acute angle is less than or equal to 25°.

14. The medical infusion pump according to claim 11, wherein the measurement path is parallel to the support plane.

15. The medical infusion pump according to claim 11, further comprising a peristaltic mechanism.

16. The medical infusion pump according to claim 15, wherein the peristaltic mechanism is configured to be covered by a front flap.

17. The medical infusion pump according to claim 16, wherein the front flap is movable to a closed position to secure or hold a section of infusion tubing in place within the medical infusion pump.

18. The medical infusion pump according to claim 17, wherein, when the front flap is moved to the closed position, the ultrasonic receiver or the ultrasonic transmitter is positioned adjacent to the front flap.

19. The medical infusion pump according to claim 16, wherein:
the ultrasonic transmitter is formed on or attached to the front flap, and
the ultrasonic receiver is fixed to the housing.

20. The medical infusion pump according to claim 16, wherein:
the ultrasonic receiver is formed on or attached to the front flap, and
the ultrasonic transmitter is fixed to the housing.
